(19) United States
(12) Patent Application Publication
(10) Pub. No.: US 2026/0240529 A1
(43) Pub. Date: Aug. 20, 2026

(54) INFORMATION PROCESSING DEVICE, OVULATION DATE ESTIMATION METHOD, AND OVULATION DATE ESTIMATION PROGRAM

(71) Applicant: SympaFit Co., Ltd., Tokyo (JP)

(72) Inventor: Taira Kajisa, Wako-shi Saitama (JP)

(73) Assignee: SympaFit Co., Ltd., Tokyo (JP)

(21) Appl. No.: 19/544,122

(22) Filed: Feb. 19, 2026

(30) Foreign Application Priority Data
Feb. 19, 2025 (JP) 2025-024914

(51) Int. Cl. A61B 10/00; G16H 50/30

(52) U.S. Cl. CPC A61B 10/0012; G16H 50/30; A61B 2010/0019

(57) ABSTRACT

An information processing device includes: an acquisition unit for acquiring information on fluctuations in glucose levels of a woman over a predetermined period; an estimation unit for estimating an ovulation date of the woman based on the fluctuation information; and a provision unit for providing ovulation date information indicating a predicted ovulation date prior to the ovulation date estimated by the estimation unit.

### CROSS REFERENCE TO RELATED APPLICATIONS

This application claims priority of Japanese (JP) Patent Application No. 2025-24914, filed on February 19, 2025, which is incorporated herein by reference in its entirety.

### TECHNICAL FIELD

This disclosure relates to an information processing device, an ovulation date estimation method, and an ovulation date estimation program for estimating an ovulation date of a woman based on fluctuations in glucose levels.

### BACKGROUND

Conventionally, there is a method for identifying an ovulation date of a woman by measuring a basal body temperature of the woman. Additionally, Japanese Patent Application Publication No. 2024-80237 discloses a technique for predicting an ovulation date by estimating the concentrations of at least two types of biological substances from a captured image of a region showing the reaction results of a specimen in an inspection instrument. The specimen is a body fluid such as blood, serum, urine, or saliva, and the biological substances are luteinizing hormone and estrogen.

Depending on the woman, hormone secretion may be unstable, and there is a possibility that the prediction by the method disclosed in Japanese Patent Application Publication No. 2024-80237 and other methods is not correct.

It could therefore be helpful to provide an information processing device, an ovulation date estimation method, and an ovulation date estimation program that estimate an ovulation date by a method different from that of Japanese Patent Application Publication No. 2024-80237.

### SUMMARY

As disclosed herein:

An information processing device according to some aspects of the present disclosure includes: an acquisition unit that acquires information on fluctuations in glucose levels over a predetermined period of a woman; an estimation unit that estimates an ovulation date of the woman based on the fluctuation information; and a provision unit that provides ovulation date information indicating a predicted ovulation date before the ovulation date estimated by the estimation unit.

In some embodiments, the estimation unit estimates, as the ovulation date of the woman, a switching timing at which the glucose level gradually increases after gradually decreasing in a predetermined period.

In some embodiments, the acquisition unit sequentially acquires the information on fluctuations in glucose levels of the woman, and the estimation unit estimates the next ovulation date by detecting that the fluctuation information of the glucose levels sequentially acquired by the acquisition unit is gradually decreasing.

In some embodiments, the estimation unit estimates an ovulation date of a woman corresponding to the fluctuation information acquired by the acquisition unit by inputting the acquired fluctuation information into a trained model that has been trained using information on fluctuations in glucose levels of a plurality of women as explanatory variables and ovulation dates corresponding to the respective pieces of fluctuation information as objective variables.

In some embodiments, the estimation unit estimates the ovulation date of the woman using information on fluctuations in glucose levels measured during the night within the predetermined period.

In some embodiments, the estimation unit averages the fluctuation information of the glucose levels measured in the predetermined period on a daily basis, and estimates the ovulation date of the woman based on the averaged fluctuation information of the glucose levels.

In some embodiments, the predetermined period is a period of at least 28 days or more.

In some embodiments, the acquisition unit also acquires body temperature information of the woman, and the estimation unit further estimates the ovulation date of the woman by also utilizing the body temperature information of the woman.

An ovulation date estimation method according to some aspects of the present disclosure causes a computer to execute steps of: acquiring information on fluctuations in glucose levels over a predetermined period of a woman; estimating an ovulation date of the woman based on the fluctuation information; and outputting the ovulation date information indicating a predicted ovulation date before the ovulation date estimated in the estimating step.

An ovulation date estimation program according to some aspects of the present disclosure causes a computer to embody functions of: acquiring information on fluctuations in glucose levels over a predetermined period of a woman; estimating an ovulation date of the woman based on the fluctuation information; and outputting the ovulation date information indicating a predicted ovulation date before the ovulation date estimated by the estimating function.

The information processing device can estimate and provide when an ovulation date of a woman will be, several days in advance, based on fluctuations in glucose levels of the woman.

### BRIEF DESCRIPTION OF THE DRAWINGS

FIG. 1 is a conceptual diagram showing an overview of an ovulation date prediction system.

FIG. 2 is a block diagram showing a configuration example of an information processing device.

FIG. 3 is a diagram schematically demonstrating a relationship between female hormones, ovulation date, and glucose levels.

FIG. 4 is a diagram schematically demonstrating glucose level fluctuations, basal body temperature, and menstrual cycle over a predetermined period.

FIG. 5 is a flowchart showing an operation example during learning of the information processing device.

FIG. 6 is a flowchart showing an operation example during estimation of the information processing device.

FIG. 7 is a graph showing a first fluctuation example of glucose levels for one month of a first woman.

FIG. 8 is a graph showing a second fluctuation example of glucose levels for one month of a second woman.

FIG. 9 is a graph showing a third fluctuation example of glucose levels for one month of a third woman.

FIG. 10 is a graph showing a fourth fluctuation example of glucose levels for one month of a fourth woman.

FIG. 11 is a diagram showing an example of a display screen for information provision.

### DETAILED DESCRIPTION

Hereinafter, an information processing device 100 according to the present disclosure will be described in detail with reference to the drawings.

### Embodiment

### Composition

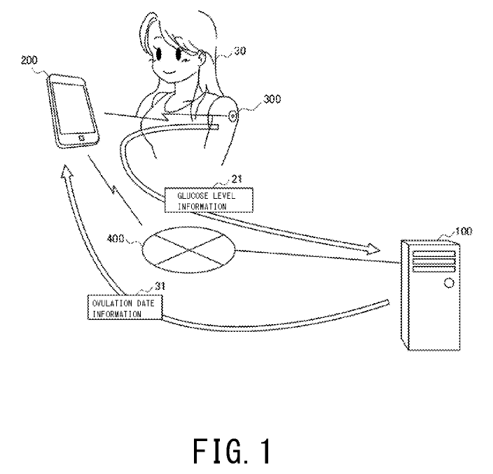

FIG. 1 is a system diagram showing an operation example of the information processing device according to some aspects of the present disclosure.

As shown in FIG. 1, a woman 30 who wishes to predict her own ovulation date lives while wearing a sensor 300 that collects information indicating her own glucose levels. The sensor 300 sequentially measures the glucose level (blood glucose level) of the woman 30 and transmits the information to the information processing terminal 200 of the woman 30 as appropriate. The sensor 300 is attached to, for example, an arm of the woman to continuously measure glucose levels, and more strictly, the sensor 300 measures interstitial fluid glucose levels that reflect blood glucose levels. Therefore, the glucose level can be substituted with a blood glucose level. Although the illustrated information processing terminal 200 shows a smartphone as an example, this may be a tablet terminal, a mobile phone, a PC (Personal Computer), a wearable terminal that can be embodied in the form of a watch, or glasses, for example. The information processing terminal 200 transmits glucose level information 21 transmitted from the sensor 300 to the information processing device 100 via the network 400 as appropriate. The information processing device 100 learns the glucose level information 21 of the woman 30, predicts the next ovulation date based on the fluctuations thereof, and transmits ovulation date information 31 to the information processing terminal 200 of the woman 30.

Thereby, the woman 30 can recognize her own ovulation date and utilize her own ovulation date, for example, for fertility treatment. Details will be described below.

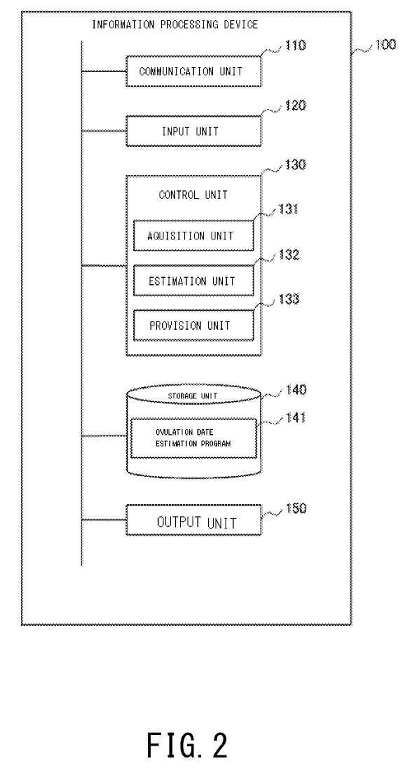

FIG. 2 is a block diagram showing a configuration example of the information processing device 100.

As shown in FIG. 2, the information processing device 100 includes a communication unit 110, a control unit 130, and a storage unit 140. Further, the information processing device 100 may include an input unit 120 and an output unit 150. The information processing device 100 may be a computer system embodied by a server device, a PC, for example.

The communication unit 110 is a communication interface that communicates with an external device via the network 400. The external device may be, for example, the information processing terminal 200 of the woman 30. The communication unit 110 receives the glucose level information 21 of the woman 30 from the information processing terminal 200 and transmits the glucose level information 21 to the control unit 130. Further, the communication unit 110 transmits the ovulation date information 31 transmitted from the control unit 130 to the information processing terminal 200. The glucose level information 21 is information in which date and time information at the time of measurement and information indicating the glucose level at that time are associated with each other. The glucose level information 21 may be transmitted from the information processing terminal 200 to the information processing device 100 for each measurement by the sensor 300, or measurement values for a predetermined period (for example, it may be for one week, but is not limited thereto, and may be for 3 days, 10 days) may be transmitted. Further, the communication unit 110 may receive information on the ovulation date input by the woman 30 from the information processing terminal 200 and transmit the information to the control unit 130.

The input unit 120 is an input interface that receives input from an operator, for example, of the information processing device 100 and transmits the input to the control unit 130. The input unit 120 may be embodied by, for example, a keyboard, a mouse, a microphone, but is not limited thereto.

The control unit 130 is a processor that controls each unit of the information processing device 100. The control unit 130 embodies functions to be performed by the information processing device 100 by executing various programs stored in the storage unit 140. That is, the control unit 130 predicts the next ovulation date of the user (woman 30) based on the glucose level information 21 of the user and transmits that information to the information processing terminal 200.

The control unit 130 may include an acquisition unit 131, an estimation unit 132, a provision unit 133, and a learning unit 134 to predict the ovulation date and transmit the information.

The acquisition unit 131 acquires the glucose level information 21 transmitted from the information processing terminal 200 of the woman 30 and received by the communication unit 110. The acquisition unit 131 stores the acquired glucose level information 21 in the storage unit 140 in association with the user identification information of the woman 30.

The estimation unit 132 predicts the next ovulation date of the woman 30 based on the glucose level information 21 of the woman 30 stored in the storage unit 140.

The estimation unit 132 predicts the ovulation date of the woman 30 using the glucose level information 21 for a predetermined period and an ovulation date estimation program 141 stored in the storage unit 140.

The estimation unit 132 predicts the ovulation date of the woman 30 based on the transition of glucose level fluctuations indicated by the glucose level information 21.

The estimation unit 132 may predict the ovulation date based on fluctuations in glucose level information during sleep, when fluctuations in glucose levels due to food intake, for example, are unlikely to occur, with respect to the glucose level information 21 of the woman 30. When the estimation unit 132 detects that the glucose level information 21 gradually decreases (decreases little by little) over several days (for example, it may be 4 days, but is not limited to 4 days, and an appropriate number of days may be determined from an average of sample data), the estimation unit 132 may estimate that several days later is the ovulation date. Further, where the ovulation date estimation program 141 estimates an ovulation date using a learning model that has learned the relationship between fluctuations in glucose levels and an ovulation date, the estimation unit 132 may input the glucose level information 21 of the woman 30 for a predetermined period into the learning model to predict the ovulation date. Further, the estimation unit 132 may perform reinforcement learning on the learning model using the glucose level information 21 of the woman 30 for a predetermined period, or perform fine-tuning using the glucose level information 21 of the woman 30 for a predetermined period, and then receive input of subsequent glucose level information of the woman 30 to predict the next ovulation date. In this disclosure, the estimation unit 132 predicts the ovulation date of the woman 30 using a learning model.

The estimation unit 132 predicts the ovulation date several days before the ovulation date (specifically, it may be about 2 or 3 days before, but is not limited thereto). The estimation unit 132 transmits the estimated ovulation date to the provision unit 133.

The provision unit 133 provides the ovulation date information 31, which predicts the next ovulation date of the woman 30 estimated by the estimation unit 132, to the information processing terminal 200 of the woman 30 via the communication unit 110.

When the ovulation date estimation program 141 predicts the ovulation date of the woman 30 using a learning model that has learned the relationship between glucose levels and the ovulation date, the learning unit 134 may relearn the learning model using the fluctuation of the glucose level of the woman 30 and information on the ovulation date in the fluctuation. That is, the learning unit 134 may learn, as teacher data, data in which the ovulation date in the fluctuation is annotated to the fluctuation of the glucose level of the woman 30 whose ovulation date is to be estimated (preferably a fluctuation for at least one menstrual cycle, but it may be a fluctuation for several days before the ovulation date including the ovulation date).

The ovulation date at this time may be the date indicated by the information on the ovulation date transmitted from the information processing terminal 200 of the woman 30. Alternatively, the learning unit 134 may learn the fluctuation of the glucose level of the woman 30 and the information on the ovulation date in the fluctuation as reference data for estimation (data for fine-tuning). By learning the relationship between the fluctuation of the glucose level and the ovulation date of the woman 30 once, the estimation of the ovulation date of the woman 30 in the next menstrual cycle can be made more accurate. Note that when the ovulation date is estimated without performing learning, the learning unit 134 may not be provided.

The storage unit 140 is a storage medium having a function of storing various programs and various data required for an operation of the information processing device 100. The storage unit 140 may be embodied by, for example, a Hard Disk Drive (HDD), a Solid State Drive (SSD), a flash memory, but is not limited thereto.

Further, the storage unit 140 may be embodied by a cloud storage. The storage unit 140 may store an ovulation date estimation program 141 that predicts the ovulation date of the woman 30 based on the glucose level information 21 of the woman 30. Further, the ovulation date estimation program 141 may predict the ovulation date depending on whether or not the fluctuation of the glucose level shows a predetermined pattern.

Further, the ovulation date estimation program 141 may predict the ovulation date of the woman 30 using a trained model that has learned the relationship between the fluctuation of glucose level information of each of a large number of women and the ovulation date. That is, the trained model may be a model that has learned teacher data in which fluctuations in glucose level information of women for a predetermined period (it may be about one month, but is not limited to one month) are used as explanatory variables and the ovulation date is used as an objective variable.

The output unit 150 is an interface having a function of outputting information designated according to instructions from the control unit 130. The output unit 150 may be embodied by, for example, a monitor, a speaker, but is not limited thereto. The output unit 150 may output information indicating an ovulation date as an example. The information processing device 100 is configured to provide the ovulation date information 31 by communication, but this may take a mode in which output is performed by the output unit 150 and the content is provided by showing the ovulation date information 31 to the woman 30.

The above is the configuration of the information processing device 100.

Note that the information processing terminal 200 is embodied by a commonly known smartphone or tablet terminal, and may be any computer system having functions similar to those information processing devices, therefore, a detailed description of the configuration is omitted here. The information processing terminal 200 may be any computer that can embody a function of communicating with the sensor 300 and receiving glucose level information from the sensor 300, a function of transmitting the glucose level information 21 to the information processing device 100, and a function of receiving the ovulation date information 31 from the information processing device 100 and outputting the ovulation date information 31.

Further, the sensor 300 is attached to an arm of the woman 30, for example, collects body fluids such as blood, saliva, and sweat of the woman 30, acquires a glucose level in the collected body fluids using a detection element, and transmits the glucose level to the information processing terminal 200.

### Relationship between Glucose Level and Ovulation Date

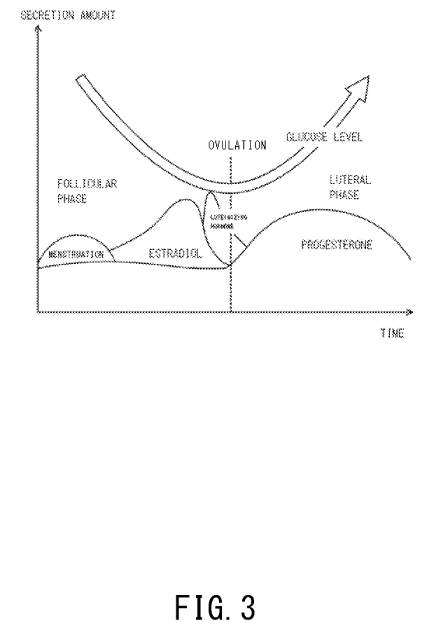

FIG. 3 is a diagram showing a relationship between glucose levels, ovulation date, and hormone secretion.

FIG. 3 is a diagram showing a relationship between fluctuations in glucose levels before and after the ovulation date and secretion of various hormones related to ovulation on a time axis on the horizontal axis, showing an example of fluctuations during one menstrual cycle.

As shown in FIG. 3, the hormones secreted in women differ before and after the ovulation date. A period before the ovulation date is called a follicular phase, and a period after the ovulation date is called a luteal phase. In women, a hormone called estradiol is gradually secreted toward the ovulation date, and secretion of estradiol reaches a peak several days before the ovulation date. Estradiol is a steroid hormone that has strong biological effects on the endometrium and myometrium, and has functions of maturing follicles, thickening the endometrium, and supporting the implantation of a fertilized egg. Note that estradiol is a type of estrogen. Further, immediately before the ovulation date, a large amount of luteinizing hormone is secreted. Luteinizing hormone is a hormone that induces ovulation and, after ovulation, luteinizes follicles and promotes the secretion of progesterone, secretion of progesterone reaches a peak before the ovulation date and continues after ovulation. Progesterone is a hormone that thickens the endometrium and prepares an environment where a fertilized egg is likely to implant, and is also a hormone that adjusts the menstrual cycle along with estradiol. Progesterone is gradually secreted in large amounts after ovulation, and the secretion amount decreases if fertilization does not occur.

In contrast, we discovered that glucose levels fluctuate before and after the ovulation date.

Specifically, as shown in FIG. 3, we discovered that glucose levels gradually decrease toward the ovulation date and gradually increase after ovulation.

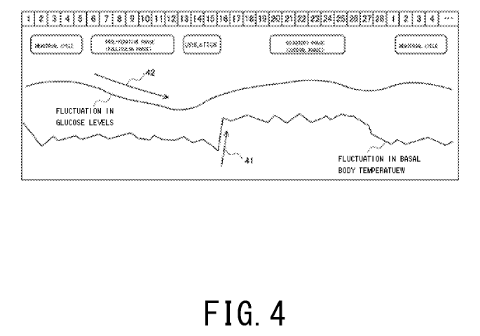

FIG. 4 is a diagram showing a relationship between glucose level fluctuations, ovulation date, menstrual cycle, and changes in basal body temperature. FIG. 4 is a diagram showing an example of changes in the menstrual cycle, glucose level fluctuations, and basal body temperature fluctuations over the number of days of the menstrual cycle.

As shown in FIG. 4, the menstrual cycle starts with the menstrual phase, goes through the proliferative phase (follicular phase), then ovulation occurs, goes through the secretory phase (luteal phase), and moves to the next menstrual phase. The menstrual cycle is said to be approximately 28 days, with the first 5 days being the menstrual phase, about the 6th to 12th days being the proliferative phase (follicular phase), ovulation occurring on the 14th or 15th day, and the period from then until the next menstrual phase being the secretory phase (luteal phase). Conventionally, ovulation was predicted by basal body temperature fluctuations.

Basal body temperature is the body temperature when in a resting state consuming only the energy necessary for life maintenance, and is usually the body temperature measured while remaining in a resting state immediately after waking up in the morning.

In the menstrual cycle, as shown in the basal body temperature fluctuations, when ovulation occurs, the basal body temperature rises sharply as indicated by arrow 41 in FIG. 4. This is due to an action of progesterone secreted after ovulation. Therefore, by measuring daily basal body temperature, an approximate ovulation date can be predicted. However, actual ovulation occurs before that, which can be seen as late for women who are trying to conceive. This is because a success rate of pregnancy is generally considered to be high two days before the ovulation date.

On the other hand, we discovered that, as shown in the glucose level fluctuations, the glucose level shows a gentle downward trend as the ovulation date approaches. That is, as shown by an arrow 42 of the glucose level fluctuation in FIG. 4, the secretion amount of the glucose level decreases as the ovulation date approaches.

Since this downward trend appears several days before the ovulation date, we discovered that if the downward trend of the glucose level or signs of the downward trend can be discovered, the ovulation date can be estimated before the ovulation date more effectively than by the basal body temperature method. The ovulation date prediction method utilizes the downward trend of the glucose level.

### Operation

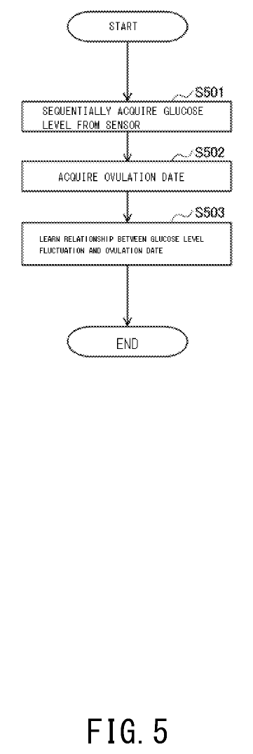

FIG. 5 is an operation example of the information processing device 100, and is a flowchart showing an operation example during learning.

As shown in FIG. 5, the communication unit 110 sequentially receives glucose level information 21 indicating the glucose level sensed by the sensor 300 from the information processing terminal 200 of the woman 30 via the network 400 (step S501).

The communication unit 110 transmits the received glucose level information 21 to the control unit 130.

Further, the communication unit 110 receives information indicating the date of the ovulation date from the information processing terminal 200 of the woman 30 (step S502). The communication unit 110 transmits the received date of the ovulation date to the control unit 130.

The learning unit 134 of the control unit 130 performs learning using information indicating the fluctuation of the glucose level for a predetermined period based on the transmitted glucose level information 21 as an explanatory variable and information indicating the transmitted ovulation date as an objective variable (step S503). The learning may be any of additional learning or reinforcement learning of a learning model as the ovulation date estimation program 141, or a process of adding it as data for reference.

Thereby, when glucose level information for the next cycle of the woman 30 is received, it becomes easier to estimate the next ovulation date of the woman 30; in other words, the accuracy of estimation can be improved.

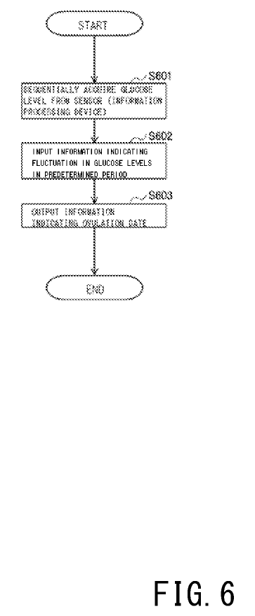

FIG. 6 is an operation example of the information processing device, and is a flowchart showing an operation example during estimation.

As shown in FIG. 6, the communication unit 110 of the information processing device 100 sequentially receives the glucose level information 21 of the woman 30 from the information processing terminal 200. The communication unit 110 transmits the received glucose level information 21 to the control unit 130. The acquisition unit 131 of the control unit 130 acquires the glucose level information transmitted from the communication unit 110 (step S601). The acquisition unit 131 transmits the acquired glucose level information to the estimation unit 132.

The estimation unit 132 connects the transmitted glucose level information to form information indicating fluctuations in glucose levels, inputs this into the trained model, and estimates the next ovulation date (step S602).

At this time, the estimation unit 132 may generate information indicating fluctuations in glucose levels using only the glucose level information of the woman 30 during sleep (during the night) among the transmitted glucose level information. As an example, the estimation unit 132 may generate information indicating fluctuations in glucose levels using only the glucose level information received between 0:00 and 6:00 to estimate the ovulation date. The estimation unit 132 transmits information indicating the estimated next ovulation date to the provision unit 133. At this time, if the ovulation date cannot be estimated from the transmitted glucose level information at the current point in time (there are no signs, or even if there are signs, the possibility that the day is the ovulation date is low), the ovulation date may not be estimated.

When the ovulation date is transmitted from the estimation unit 132, the provision unit 133 (output unit 150) transmits the ovulation date to the information processing terminal 200 that transmitted the glucose level information via the communication unit 110 (step S604). When the information indicating the ovulation date is received from the information processing device 100, the information processing terminal 200 displays information indicating the ovulation date. The woman 30 looks at the information and can recognize her own ovulation date, and can utilize the information, for example, for fertility treatment.

The above is the description of the ovulation date estimation process.

### Specific Examples of Relationship Between Glucose Level Fluctuations and Ovulation Date

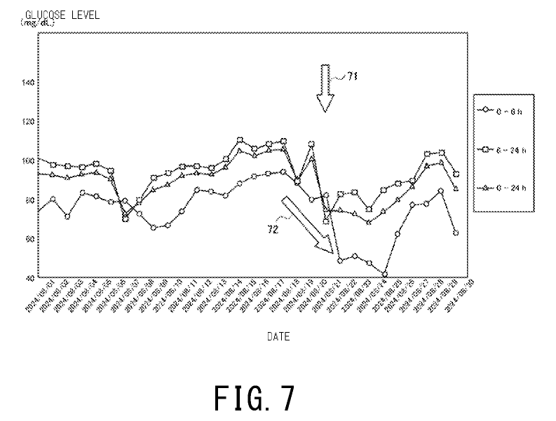

FIGS. 7 to 10 are graphs showing examples in which glucose levels were measured using four women as subjects.

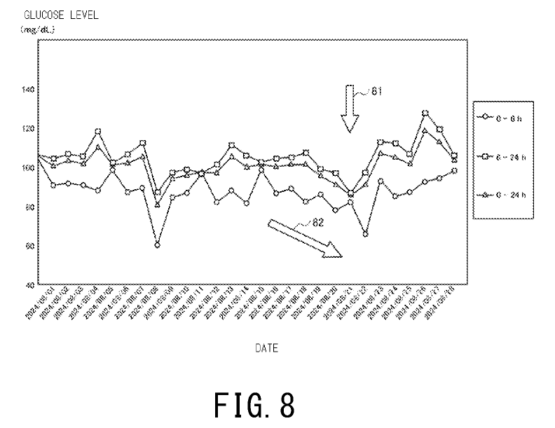

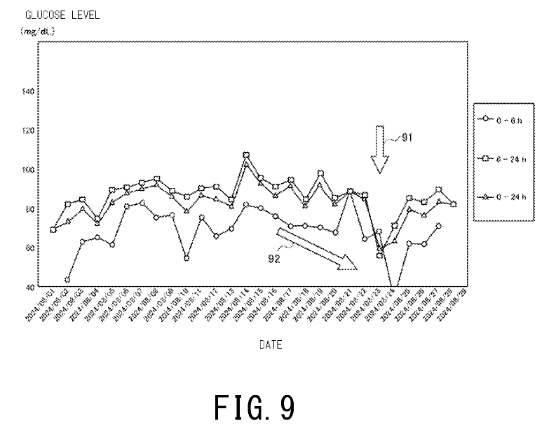

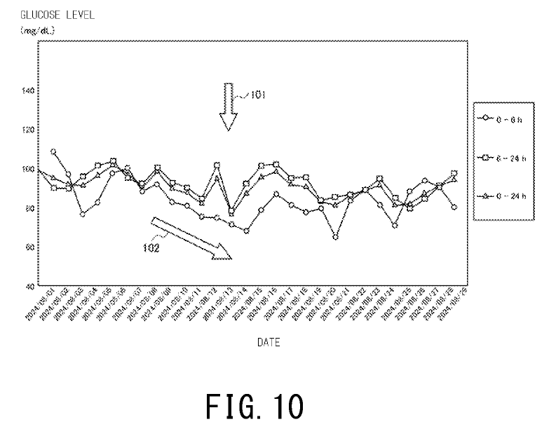

FIG. 7 is a graph showing fluctuations in glucose levels measured for one month for a first woman. FIG. 8 is a graph showing fluctuations in glucose levels measured for one month for a second woman. FIG. 9 is a graph showing fluctuations in glucose levels measured for one month for a third woman. FIG. 10 is a graph showing fluctuations in glucose levels measured for one month for a fourth woman.

In the graphs shown in FIGS. 7 to 10, the horizontal axis indicates the date, and the vertical axis indicates the secretion amount of the glucose level. Glucose level plot in each day is a plot of the average value of the glucose secretion amount in each time period. The circles in the plots indicate the average value of glucose levels measured between 0:00 and 6:00 within a day. Further, the squares in the plots indicate the average value of glucose levels measured between 6:00 and 24:00 within a day. Then, the triangles in the plots indicate the average value of glucose levels measured from 0:00 to 24:00, i.e., in one day.

An arrow 71 in the graph shown in FIG. 7 indicates the ovulation date. That is, the first woman had August 20, 2024, as the ovulation date. From the fluctuation of the circles in the plot in FIG. 7, it can be confirmed that the average value of the glucose levels gradually decreased from August 16 to August 20, as shown by arrow 72. It can be understood that the plots of squares and triangles include glucose level information when the first woman is leading her daily life, i.e., information including increases in glucose levels due to eating and drinking, and are therefore not suitable information for looking at qualitative glucose level fluctuations.

An arrow 81 in the graph shown in FIG. 8 indicates the ovulation date. That is, the second woman had August 21, 2024, as the ovulation date. From the fluctuation of the circles in the plot in FIG. 8, as shown by arrow 82, although there are some upward and downward fluctuations, it can be confirmed that the average value of the glucose levels gradually decreased from August 15 to August 22 overall. It can be understood that the plots of squares and triangles include glucose level information when the second woman is leading her daily life, i.e., information including increases in glucose levels due to eating and drinking, and are therefore not suitable information for looking at qualitative glucose level fluctuations.

An arrow 91 in the graph shown in FIG. 9 indicates the ovulation date. That is, the third woman had August 23, 2024, as the ovulation date. From the fluctuation of the circles in the plot in FIG. 9, it can be confirmed that the average value of the glucose levels gradually decreased from August 14 to August 23, as shown by arrow 92. It can be seen that there was a large increase on August 21, but this was due to the woman having a snack in the middle of the night. It can be understood that the plots of squares and triangles include glucose level information when the third woman is leading her daily life, i.e., information including increases in glucose levels due to eating and drinking, and are therefore not suitable information for looking at qualitative glucose level fluctuations.

An arrow 101 in the graph shown in FIG. 10 indicates the ovulation date. That is, the fourth woman had August 13, 2024, as the ovulation date. From the fluctuation of the circles in the plot in FIG. 10, it can be confirmed that the average value of the glucose levels gradually decreased from August 7 to August 13, as shown by arrow 102. The fourth woman has a more night-oriented lifestyle than other women, and this can be seen from the fact that glucose levels measured between 0:00 and 6:00 sometimes exceed those of other average values. However, even in such a woman, a gradual decrease trend in glucose levels can be seen from several days before the ovulation date.

As seen from these results, the secretion amount of glucose gradually decreases in any of the women starting several days before the ovulation date. Therefore, it is possible to sequentially acquire the glucose level information of the woman 30, detect that the fluctuation of the glucose level has a gradual decrease trend, and estimate that the ovulation date will be several days later.

### Output Example

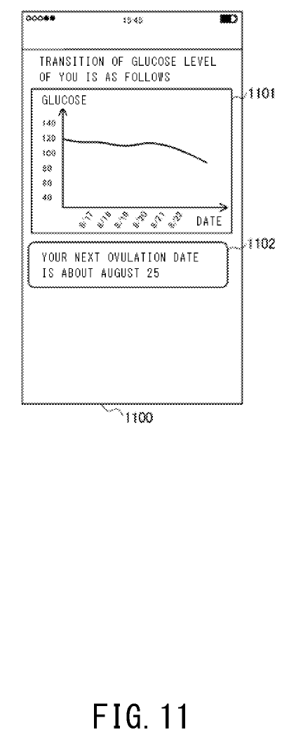

FIG. 11 is a diagram showing an output example by the information processing terminal 200. The output example shown in FIG. 11 is merely an example, and output may be performed in modes other than this. An output example 1100 provided by the information processing device 100 and displayed by the information processing terminal 200 shown in FIG. 11 includes at least information 1102 indicating the next ovulation date estimated by the information processing device 100. Further, as shown in FIG. 11, a graph 1101 showing fluctuations in glucose levels of the woman 30, which is the basis for predicting the ovulation date, may also be output. In addition to this, where the woman 30 is measuring a basal body temperature, fluctuations in the basal body temperature may also be output together.

### Conclusion

The information processing device 100 can estimate an ovulation date several days before the ovulation date by looking at actual fluctuations in glucose levels of the woman 30. Therefore, the information processing device 100 can be useful for fertility treatment for the woman 30.

### Supplements

The information processing device is not limited to the above embodiment, and each configuration may be embodied by other methods. Various modifications will be described below.

(1) The information processing device 100 receives the glucose level information 21 from the information processing terminal 200 of the woman 30, and transmits and displays the ovulation date information 31 predicting the next ovulation date of the woman 30 on the information processing terminal 200 of the woman 30, thereby notifying the woman 30 of her predicted ovulation date. However, this is not limited thereto. The prediction of the ovulation date shown may be embodied by a native application operating on the information processing terminal 200 alone. The native application may embody each function of the information processing device 100 by an application. Further, the prediction of the ovulation date shown may be embodied by cooperation between an application operating on the information processing terminal 200 and the information processing device 100. That is, the prediction of the ovulation date shown may be embodied by any of a browser type, a native application type, and a client-server type application.

(2) The information processing device 100 predicts the next ovulation date after once learning the fluctuation of the glucose level of the woman 30, but the information processing device 100 may predict the next ovulation date without learning. That is, the process shown in FIG. 5 may be omitted. Since the learning model of the information processing device 100 is a model that has learned the relationship between fluctuations in glucose levels and ovulation dates of many women, it is also possible to predict the ovulation date of the woman 30 by providing glucose level information. However, the prediction accuracy of the ovulation date can be made higher if the glucose level information of the woman 30 is learned in advance.

(3) The ovulation date is estimated using the glucose level information of the woman 30 acquired from 0:00 to 6:00, but it is preferable that this is glucose level information measured during sleep of the woman 30, and it is not limited to that acquired from 0:00 to 6:00. Thus, the information processing terminal 200 may receive input of information on when the woman 30 went to bed and when she woke up and transmit the information to the information processing device 100. Then, the information processing device 100 may estimate the ovulation date for each day using only the glucose level information acquired between the transmitted bedtime and wake-up time.

(4) The information processing device 100 estimates the ovulation date based on fluctuations in glucose levels, but information other than glucose levels may also be utilized as information serving as a basis for determination.

For example, the information processing device 100 may estimate the ovulation date using information on the basal body temperature of the woman 30 in addition to the glucose level, as in the conventional instance. Thus, the information processing device 100 may estimate the ovulation date using a trained model that has learned the relationship between fluctuations in basal body temperature of the woman 30 and the ovulation date. For example, in utilizing basal body temperature, the basal body temperature may be used as information for reinforcing the ovulation date predicted using glucose levels. Reinforcing means, for example, that when estimation based on basal body temperature deviates from estimation based on glucose levels, the ovulation date estimated based on glucose levels may be corrected to an ovulation date estimated based on basal body temperature, or corrected in the direction of the ovulation date estimated based on basal body temperature.

(5) As a method for estimating and providing an ovulation date of a woman based on glucose levels in an information processing device, the processor of the information processing device 100 executes an ovulation date estimation program, for example, to perform estimation and provision, however, this may be embodied by a logic circuit (hardware) or a dedicated circuit formed in an integrated circuit (IC) chip, Large Scale Integration (LSI), for example, in the device.

Further, these circuits may be embodied by one or a plurality of integrated circuits, and the functions of the plurality of functional units shown in the above embodiment may be embodied by one integrated circuit. LSIs may also be called VLSIs, Super LSIs, Ultra LSIs, for example, depending on the degree of integration.

The ovulation date estimation program may be recorded on a processor-readable recording medium, and as the recording medium, a “non-transitory tangible medium”, for example, a tape, a disk, a card, a semiconductor memory, a programmable logic circuit can be used. Further, the ovulation date estimation program may be supplied to the processor via an arbitrary transmission medium (communication network, broadcast wave, for example) capable of transmitting the ovulation date estimation program. That is, for example, a configuration may be adopted in which an ovulation date estimation program is downloaded from a network and executed using an information processing device such as a smartphone. This disclosure can also be embodied in the form of a data signal embedded in a carrier wave, in which the ovulation date estimation program is embodied by electronic transmission.

The ovulation date estimation program can be implemented using, for example, a script language such as ActionScript or JavaScript (registered trademark), or an object-oriented programming language such as Objective-C, Java (registered trademark), C++, Python (registered trademark), or R language.

(6) The various configurations shown in the above embodiment and supplements may be combined as appropriate.

What is claimed is:

1. An information processing device comprising:
an acquisition unit for acquiring fluctuation information of glucose levels of a woman over a predetermined period;
an estimation unit for estimating an ovulation date of the woman based on the fluctuation information; and
a provision unit for providing ovulation date information indicating a predicted ovulation date prior to the ovulation date estimated by the estimation unit.

2. The information processing device according to claim 1, wherein the estimation unit estimates a switching timing at which the glucose levels gradually increase after gradually decreasing during a predetermined period as the ovulation date of the woman.

3. The information processing device according to claim 2, wherein the acquisition unit sequentially acquires the fluctuation information of glucose levels of the woman, and the estimation unit detects that the fluctuation information of the glucose level sequentially acquired by the acquisition unit gradually decreases, thereby estimating the next ovulation date.

4. The information processing device according to claim 1, wherein the estimation unit estimates an ovulation date of a woman corresponding to the fluctuation information acquired by the acquisition unit by inputting the acquired fluctuation information into a trained model that has been trained using information on fluctuations in glucose levels of a plurality of women as explanatory variables and ovulation dates corresponding to the respective pieces of fluctuation information as objective variables.

5. The information processing device according to claim 1, wherein the estimation unit estimates the ovulation date of the woman using information on fluctuations in glucose levels measured during a night within the predetermined period.

6. The information processing device according to claim 5, wherein the estimation unit averages the information on fluctuations in glucose levels measured during the predetermined period on a daily basis and estimates the ovulation date of the woman based on the averaged information on fluctuations in glucose levels.

7. The information processing device according to claim 1, wherein the predetermined period is at least 28 days or longer.

8. The information processing device according to claim 1, wherein the acquisition unit also acquires body temperature information of the woman, and the estimation unit further estimates the ovulation date of the woman using body temperature information of the woman.

9. A method for estimating an ovulation date, the method causing a computer to execute steps of:
acquiring information on fluctuations in glucose levels of a woman over a predetermined period;
estimating an ovulation date of the woman based on the fluctuation information; and
outputting the ovulation date information indicating the predicted ovulation date before the ovulation date estimated in the estimating step.

10. A non-transitory computer-readable medium storing a program for estimating an ovulation date, the program causing a computer to execute the functions of:
acquiring information on fluctuations in glucose levels of a woman over a predetermined period;
estimating an ovulation date of the woman based on the fluctuation information; and
outputting the ovulation date information indicating the predicted ovulation date before the ovulation date estimated in the estimating function.
